(19) United States
(12) Patent Application Publication
(10) Pub. No.: US 2002/0002364 A1
(43) Pub. Date: Jan. 3, 2002

(54) Osmotic device for the continuous release of active compounds into the fluids of the gastrointestinal tract

(76) Inventor: Karsten Cremer, Bonn (DE)

Correspondence Address: WENDEROTH, LIND & PONACK, Suite 800, 2033 "K" Street N.W., Washington, DC, 20006, US

(21) Appl. No.: 09/938,545

(22) Filed: Aug. 27, 2001

Related U.S. Application Data
(63) Continuation of application No. 09/011,072, filed on Jan. 30, 1998, now abandoned.
Application No. 09/011,072 is a 371 of international application No. PCT/EP96/04117, filed on Sep. 20, 1996.

(30) Foreign Application Priority Data
Oct. 5, 1995 (DE) 195 37 090.2

(51) Int. Cl. A61K 9/22

(52) U.S. Cl. 604/892.1; 604/93.01; 604/891.1

(57) ABSTRACT

An osmotic device for the controlled release of active compounds into the fluid of the gastrointestinal tract through an outlet opening arranged in the outer membrane of the device is characterized in that the outlet opening, compared with areas of the device designed to maintain a distance relative to the surface of the mucous membrane, is arranged in a position of the external membrane at a distance from the surface of the mucous membrane which, relatively thereto, is further away.

### DESCRIPTION

The invention relates to an osmotic device for the controlled release of active compounds into the fluids of the gastrointestinal tract through an outlet opening arranged in the outer membrane of the device.

Such osmotic devices represent one category of the so-called therapeutic systems for peroral administration (K. E. Bauer et al., Pharmazeutische Technologie, pp. 554-561, Thieme Verlag, 1986). They are able to release active compounds in a substantially more controlled manner than, for example, conventional delayed-release pharmaceutical forms.

The mode of operation of an osmotic device of this type can be illustrated as exemplified by the simplest possible construction, the single chamber system. One of these consists of an osmotically active core containing the active compound, which is coated with a semi-permeable polymer membrane. The polymer membrane has a small opening through which the active compound is released into the surrounding fluid. As a rule, the core of the device is pressed as a tablet from a mixture of active compound and auxiliaries. The polymer membrane is produced, for example, by a spraying process with which the polymer is applied to the core with further auxiliaries as a solution or dispersion and then dried. The outlet opening in the polymer membrane is produced by a laser beam.

After the peroral administration of an osmotic therapeutic system in the intestinal tract, fluid diffuses from this through the polymer membrane into the active compound-containing core. The active compound and/or an osmotically active auxiliary intended for this purpose begin to dissolve therein. By this means, an increased osmotic pressure compared with the external medium results in the interior of the device, which results in the active compound-containing solution being pushed outwards through the outlet opening. Because the rediffusion of liquid through the polymer membrane into the core takes place continuously, pressure equalization due to escape of active compound takes place with great uniformity, as long as active compound is present in the core. The rate of active compound release can be advantageously adjusted by means of the composition, condition and distension of the membrane and also by means of the solubility of the constituents of the core.

Peroral osmotic therapy systems, however, can have the disadvantage of an injury potential for the intestinal mucous membrane in addition to the advantage of control of the release rate. In this case, the injury potentials of the active compounds and auxiliaries, which are often released in highly concentrated form, can be significantly increased as a result of the focusing through the small outlet opening, so that the intestinal wall is seriously injured at certain points.

An osmotic device only employed therapeutically in Germany had to be taken off the market again in 1983 on account of the severe intestinal wall injuries produced by it. The injury potential both of the active compound indomethacin contained in the product and the osmotically active auxiliary potassium chloride was particularly high.

However, even when using osmotic therapy systems which are less injurious to mucous membranes, it cannot be excluded that lesions are caused, in particular in previously injured patients, by a focusing effect of the outlet opening.

Moreover, it would be desirable also to be able to administer those active substances in osmotic systems which admittedly have a comparatively low potential for injuring the mucous membrane, but only to such an extent which still makes an oral administration appear sensible after weighing up the benefits and risks. These substances can be administered according to the present state of the art in conventional oral administration forms, but not in peroral osmotic therapy systems.

The invention is based on the object of reporting an osmotic device for the controlled release of active compounds into the fluids of the gastrointestinal tract through an outlet opening arranged in the external membrane of the device, of the type mentioned in the precharacterizing clause of claim 1 and of improving this in comparison with the prior art to the extent that the danger of an injury to the intestinal mucous membrane is avoided to the greatest extent possible and even in the case of previously injured patients no lesions are caused.

The object is achieved by a positioning according to the invention of the outlet opening for active compound in combination with a special shaping of the osmotic device according to claim 1.

Advantageously, in this case the device is arranged such that the outlet opening, compared with areas of the device designed to maintain a distance in relation to the mucous membrane surface, is arranged in a position of the external membrane at a distance from the surface of the mucous membrane which, relatively thereto, is further away.

In this connection, the device can have hollows, concave curvatures, curved or angled axes, circular shaping or other design features in which direct or close contact of the outlet opening with the surface of the mucous membrane lining the gastrointestinal tract is not possible. The invention thus provides for an outlet opening for active compound solution to be compulsorily positioned at a distance from surface regions of the intestinal wall by maintaining a distance between the outlet opening and the part of the adjacent intestinal tract. This distance results in the active compound solution not meeting a small area of the intestinal mucous membrane corresponding to the outlet opening in the same high concentration in which it passes through the outlet opening, but meeting a larger area thereof, and also in dilute form. After administration of a device according to the invention, active compounds or auxiliaries having a potential to injure the mucous membrane reach the gastrointestinal mucous membrane only after prior dilution and can only damage the latter significantly less-if at all-than conventional osmotic therapy systems. The abovementioned advantages of the osmotic therapy system are therefore retained by a device according to the invention, while the pharmaceutical safety on its use is significantly improved.

Further details, features and advantages of the invention follow from the explanation below of some exemplary embodiments shown schematically in the drawings.

These show:

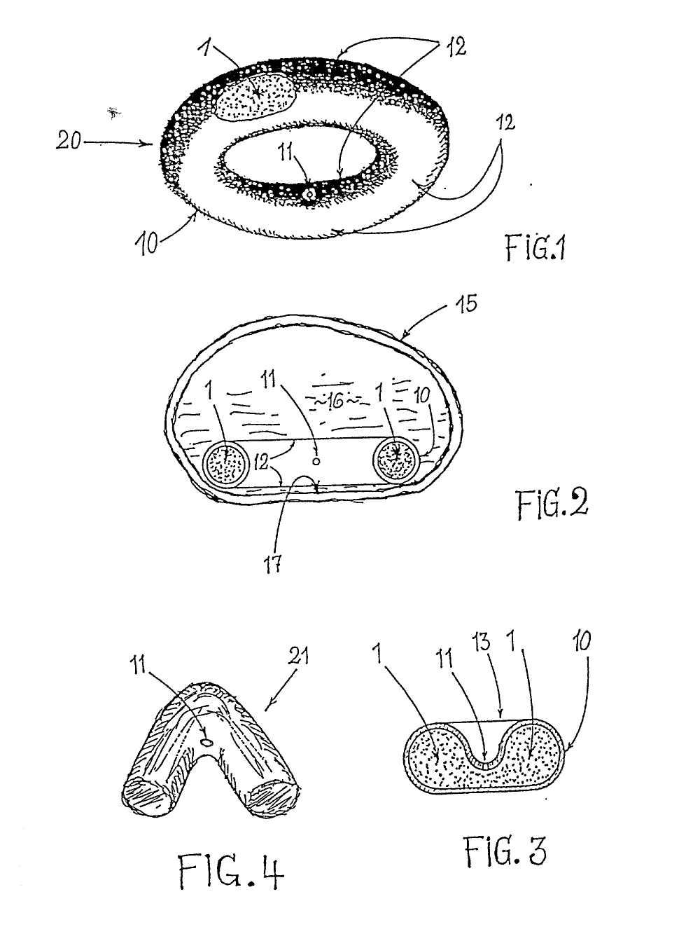

FIG. 1: in perspective a device in the form of a ring with circular or approximately circular cross-section;

FIG. 2: in section a device lying inside the intestinal wall;

FIG. 3: in section another embodiment of the device;

FIG. 4: in perspective view a further embodiment of the device.

The overall view of FIGS. 1 to 4 shows osmotic devices for the treatment of the mucous membrane (17) of the gastrointestinal tract by means of osmotically regulated, controlled release of active compound into the fluid (16) of the gastrointestinal tract through an outlet open ing (11) arranged in the external membrane (10) of the device.

The device consists of an osmotic core (1) containing the active compound, which is coated with a semi-permeable polymer membrane (10). The polymer membrane (10) has an outlet opening (11) having a comparatively small diameter, through which active compound dissolved in the fluid of the gastrointestinal tract is delivered to the surrounding fluid (16). As a rule, the core (1) of the device is pressed as a tablet f from a mixture of active compound and auxiliaries. The polymer membrane (10) can be produced, for example, by a spraying process with which the polymer is applied to the core (1) as a solution or dispersion, if appropriate with further auxiliaries, and then dried. The outlet opening (11) in the polymer membrane (10) is produced by a laser beam.

In FIG. 1, an exemplary embodiment of the osmotic device is shown which is designed in the form of a ring (20) having a circular or approximately circular cross-section, the outlet opening (11), for example when the device is lying on the surface (17) of the mucous membrane of an intestinal wall (15) (FIG. 2), being arranged on the internal wall area of the ring (20) at a distance from the surface (17) of the mucous membrane. FIG. 1 furthermore shows the known construction of a core (1) containing active compound and auxiliary in a coating by the membrane (10), the outlet opening (11), compared with areas (12) of the device designed to maintain a distance relative to the surface (17) of the mucous membrane, being arranged in a position of the external membrane (10) at a distance from the surface of the mucous membrane which, relatively thereto, is further away.

In FIG. 2, the position of the device is shown in section inside a section of the intestine shown with the intestinal wall (15). The device consisting of core (1) and coating polymer membrane (10) has the outlet opening (11), compared with areas (12) designed to maintain a distance in relation to the surface (17) of the mucous membrane, in a position of the external membrane (10) at a distance from the surface (17) of the mucous membrane which, relatively thereto, is further away.

The intestinal fluid inside the gastrointestinal tract is indicated by (16). This can diffuse through the polymer membrane (10) and then dissolves portions of the active compound or of the added auxiliaries in a continuous process, an osmotic pressure resulting in the interior of the device which allows the active compound- and auxiliary-containing solution to escape through the outlet opening (11) in a manner known per se.

The teaching of the invention, that the outlet opening (11), as a result of the particular design of the device, is arranged at a distance from the surface (17) of the mucous membrane, whatever way the device is lying on the surface (17) of the mucous membrane of the intestinal wall (15), can understandably be realized by a large number of appropriately designed exterior forms of the device.

Apart from the form of a ring, the form of an ellipse, an ellipsoid or of sections of a ring or an ellipse or an ellipsoid are also possible as well as polygonal forms and the like.

For example, the outlet opening (11), as FIG. 3 shows, can be arranged at the bottom of a hollow between distance-maintaining elevations (13). The outlet opening (11), however, can also be arranged in a concave area of the device. Furthermore, for example, the device according to FIG. 4 can have the form of a body (21) with a curved or angled axis, the outlet opening (11) then being arranged in the inner area of the curve or of the angle. The device is uncomplicated, can be prepared by economical means and optimally fulfils the object mentioned at the outset.

1. Osmotic device for the controlled release of active compounds into the fluid (16) of the gastrointestinal tract through an outlet opening (11) arranged in the outer membrane (10) of the device, characterized in that the outlet opening (11), compared with areas (12, 13) of the device maintaining a distance relative to the surface (17) of the mucous membrane, is arranged in a position of the external membrane (10) at a distance from the surface (17) of the mucous membrane which, relatively thereto, is further away.

2. Device according to claim 1, characterized in that it is designed in the form of a ring (20) having a circular or approximately circular cross-section and the outlet opening (11) is arranged on the inner wall area of the ring (20).

3. Device according to claim 1 or 2, characterized in that it is designed in the form of a ring segment.

4. Device according to claim 1, characterized in that it is designed in the form of an ellipse or of an ellipsoid with a circular or approximately circular cross-section and the outlet opening (11) is arranged on the inner wall area of the ellipse or of the ellipsoid.

5. Device according to claim 4, characterized in that it is designed in the form of a segment of the ellipse or of the ellipsoid.

6. Device according to claim 1, characterized in that the outlet opening (11) is arranged at the bottom of a hollow.

7. Device according to claim 1, characterized in that the outlet opening (11) is arranged in a concave area thereof.

8. Device according to claim 1, characterized in that this has the form of a body (21) having a curved or angled axis and the outlet opening (11) is arranged in the inner area of the curve or of the angle.
